(19) United States
(12) Patent Application Publication
(10) Pub. No.: US 2022/0059194 A1
(43) Pub. Date: Feb. 24, 2022

(54) 3D PHARMACOPHORE MODEL FOR THE RAPID COMPUTATIONAL SCREENING OF SARS-COV-2 MODULATORS AND COMPOSITIONS AND METHODS THEREOF

(71) Applicant: FluoroMe, Inc.,, Houston, TX (US)

(72) Inventors: Dimitrios VLACHAKIS, Houston, TX (US); Charles NEBLETT, Houston, TX (US); Maria KASTI, Houston, TX (US)

(21) Appl. No.: 17/409,433

(22) Filed: Aug. 23, 2021

(30) Foreign Application Priority Data
Aug. 24, 2020 (GR) 20200100511
Aug. 24, 2020 (GR) 20200100512

(51) Int. Cl. G16C 20/40; A61K 45/06

(52) U.S. Cl. CPC G16C 20/40; G16C 20/50; A61K 45/06

(57) ABSTRACT

The invention encompasses compositions and compounds for inhibiting CoV2 Spike GP and human ACE2 proteins and a 3D pharmacophore model described herein provides the means for rapid, high-throughput virtual screening of potential anti-CoV2 modulators thus facilitating, optimizing and speeding up the search for the discovery of a potent anti-COVID-19 agent and methods of treatment and prevention thereof.

### CROSS REFERENCE TO RELATED APPLICATIONS

This application claims the benefit of Greek patent application number 22-0003490898, filed Aug. 24, 2020, and Greek patent application number 22-0003490903, filed Aug. 24, 2020, each of which is incorporated herein by reference in its entirety.

### FIELD OF THE INVENTION

The invention encompasses compositions and compounds for inhibiting CoV2 Spike GP and human ACE2 proteins and a 3D pharmacophore model described herein provides the means for rapid, high-throughput virtual screening of potential anti-CoV2 modulators thus facilitating, optimizing and speeding up the search for the discovery of a potent anti-COVID-19 agent and methods of treatment and prevention thereof.

### BACKGROUND OF THE INVENTION

Coronaviruses (CoVs) infect human and animals and cause varieties of diseases, including respiratory, enteric, renal, and neurological diseases. They are classified into four genera, alpha-CoV, beta-CoV, gamma-CoV, and delta-CoV2. In 2002-2003, severe acute respiratory syndrome coronavirus (SARS-CoV), a lineage B beta-CoV, emerged from bat and palm civet and infected over 8000 people and caused about 800 deaths. In 2012, Middle East respiratory syndrome coronavirus (MERS-CoV), a lineage C beta-CoV, was discovered as the causative agent of a severe respiratory syndrome in Saudi Arabia, with 2494 confirmed cases and 858 deaths, it remains endemic in Middle East, and dromedary camel is considered as the zoonotic reservoir host of MERS-CoV. At the end of 2019, a novel coronavirus, named SARS-CoV-2, was found in patients with severe pneumonia in Wuhan, China. Viruses were isolated from patients and sequenced. Phylogenetical analysis revealed that it is a lineage B beta-CoV and closely related to a SARS-like (SL) CoV, RaTG13, discovered in a cave of Yunnan, China in 2013. They share about 96% nucleotide sequence identities, suggesting that SARS-CoV-2 might have emerged from a Bat SL-CoV. However, the intermediate host or whether there is an intermediate host remains to be determined.

CoV uses its spike glycoprotein (S), a main target for neutralization antibody, to bind its receptor, and mediate membrane fusion and virus entry. Each monomer of trimeric S protein is about 180 kDa, and contains two subunits, S1 and S2, mediating attachment and membrane fusion, respectively. In the structure, N- and C-terminal portions of S1 fold as two independent domains. N-terminal domain (NTD) and C-terminal domain (C-domain). Depending on the virus, either NTD or C-domain can serve as the receptor-binding domain (RBD). While RBD of mouse hepatitis virus (MHV) is located at the NTD14, most of other CoVs, including SARS-CoV and MERS-CoV use C-domain to bind their receptors. MHV uses mouse carcinoembryonic antigen related cell adhesion molecule 1a (mCEACAM1a) as the receptor20, and the receptors for SARS-CoV and MERS-CoV are human angiotensin-converting enzyme 2 (hACE2) and human dipeptidyl peptidase 4 (hDPP4), respectively. While S proteins of SARS-CoV-2 share about 76% and 97% of amino acid identities with SARS-CoV and RaTG13, respectively, the amino acid sequence of potential RBD of SARS-CoV-2 is only about 74% and 90.1% homologous to that of SARS-CoV and RaTG13, respectively.

The most important structural proteins of CoV are spike (S) protein (trimeric), membrane (M) protein, envelop (E) protein, and the nucleocapsid (N) protein. Some of the viruses such as beta-CoVs also have hemagglutinin esterase (HE) glycoprotein. The RNA genome of CoV has seven genes that are conserved in the order: ORF1a, ORF1b, S, OEF3, E, M, N in 5′ to 3′ direction. The two-third part of the RNA genome is covered by the ORF1a/b, which produces the two viral replicase proteins that are polyproteins (PP1a and PP1ab). Sixteen mature nonstructural proteins (NSPs) arise from further processing of these two PPs. These NSPs take part in different viral functions including the formation of the replicase transcriptase complex. The remaining genome part of the virus encodes the mRNA which produces the structural proteins, i.e., spike, envelope, membrane, and nucleocapsid, and other accessory proteins. Another important envelop-associated protein which is expressed by only some strains of CoV is the HE protein. Structure. Function, and Evolution of Coronavirus Spike Proteins. See Li. F., Structure, Function, and Evolution of Coronavirus Spike Proteins., Annu Rev Virol. 2016 Sep. 29; 3(1):237-261

CoV attaches to the target cells with the help of spike protein-host cell protein interaction (angiotensin converting enzyme-2 [ACE-2] in SARS-CoV and dipeptidyl peptidase-4 [DPP-4] in MERS-CoV). After the receptor recognition, the virus genome with its nucleocapsid is released into the cytoplasm of the host cell. The viral genome contains ORF1a and ORF1b genes, which produce two polyproteins (PPs) that are pp1a and pp1b, which help to take command over host ribosomes for their own translation process. Both pp1a and pp1b take part in the formation of the replication transcription complex. After processing of PP by protease, it produces 16 NSPs. All NSPs have their own specific functions such as suppression of host gene expression by NSP1 and NSP2, formation of a multidomain complex by NSP3, NSP5 which is a M protease which is involved in replication, NSP4 and NSP6 which are transmembrane (TM) proteins, NSP7 and NSP8 which act as a primase, NSP9—a RNA-binding protein, the dimeric form of which is important for viral infection. Induction of disturbance to the dimerization of NSP9 can be a way to overcome CoV infection. NSP10 acts as a cofactor for the activation of the replicative enzyme. NSP12 shows RNA-dependent RNA polymerase activity, NSP13 shows helicase activity, NSP14 shows exoribonuclease activity, NSP15 shows endoribonuclease activity, and NSP16 has methyltransferase activity. All NSPs have an important role in replication and transcription.

Synthesized proteins such as M, E, and S are entered into the endoplasmic reticulum (ER)-Golgi intermediate compartment (ERGIC) complex and make the structure of viral envelope. On the other hand, the replicated genome binds to N protein and forms the ribonucleoprotein (RNP) complex. The outer cover is formed by the M, E. and S proteins. Finally, the virus particle comes out of the ERGIC by making a bud-like structure. These mature virions form a vesicle, which fuses with the plasma membrane and releases the virus particles into the extracellular region. On infection, the SARS-CoV and MERS-COV cause a surge of pro-inflammatory cytokines and chemokines, which cause damage to lung tissue, deterioration of lung function, and then finally lung failure in some cases.

The instant invention encompasses a tailor-made pharmacophore model for CoV2 Spike GP and human ACE2 proteins, 3D pharmacophore model providing the means for rapid, high-throughput virtual screening of potential anti-CoV2 modulators thus facilitating, optimizing and accelerating the search for the discovery of a potent anti-COVID19 agent.

### SUMMARY OF THE INVENTION

The invention encompasses a tailor-made Pharmacophore model based on the anatomy and physicochemical properties of the CoV2 Spike GP and human ACE2 interaction interface.

The invention also encompasses the identification of compounds and their possible 3D conformations in the vicinity of the CoV2 Spike GP and human ACE2 interaction interface and to provide the means required to establish a 3D-pharmacophore model that accurately screens for specific anti-CoV2 high specific agents.

In certain embodiments, this correlation was achieved by designing a tailor made pharmacophore model based on the anatomy and physicochemical properties of the CoV2 Spike GP and human ACE2 interaction interface.

In certain embodiments, the invention encompass compositions and method including a tailor-made 3D pharmacophore model for the CoV2 Spike GP and human ACE2 for the rapid and efficient in silico screening of potential anti-CoV2 modulators.

In certain embodiments, the invention encompasses a 3D pharmacophore model consisting of the 5 pharmacophoric annotation points (PAP) that are described in FIG. 2.

In certain embodiments, the method of 3D pharmacophore model for drug design and development is based only on PAP 1, PAP 2, PAP 3 or PAP 1, PAP 2, PAP 4 or PAP 1, PAP 2, PAP 5 or PAP 1, PAP 2, PAP 3, PAP 4 or PAP 1, PAP 2, PAP 3, PAP 5 or PAP 1, PAP 2, PAP 4, PAP 5.

In certain embodiments, the 3D pharmacophore model for drug development relates to the designing of agents as inhibitors or either CoV2 Spike GP or human ACE2.

In other embodiments, the 3D pharmacophore model for drug design and development relates to the designing of agents either as small low-molecular weight or polymer-like anti-CoV2 agents.

### BRIEF DESCRIPTION OF THE FIGURES

FIG. 1 illustrates the CoV2 Spike GP and ACE-2 biological system with the tailor-made 3D pharmacophore model in the three-dimensional space of the two proteins

FIG. 2 illustrates the tailor-made 3D pharmacophore model for the CoV2 Spike GP and human ACE2 interaction interface for the rapid and efficient in silico screening of potential anti-CoV2 modulators.

FIG. 3 illustrates the life cycle of CoV in host cells. The S proteins of CoV binds to cellular receptor angiotensin-converting enzyme 2 (ACE2), which is followed by entry of the viral RNA genome into the host cell and translation of structural and non-structural proteins (NSP) follows. ORF1a and ORF1b are translated to produce pp1a and pp1ab polyproteins, which are cleaved by the proteases that are encoded by ORF1a to yield 16 non-structural proteins. This is followed by assembly and budding into the lumen of the ERGIC (Endoplasmic Reticulum Golgi Intermediate Compartment). Virions are then released from the infected cell through exocytosis.

### DETAILED DESCRIPTION OF THE INVENTION

The term “acyl” is art-recognized and refers to a group represented by the general formula hydrocarbylC(O)—, preferably alkylC(O)—.

The term “acylamino” is art-recognized and refers to an amino group substituted with an acyl group and may be represented, for example, by the formula hvdrocarbylC(O)NH—.

The term “acyloxy” is art-recognized and refers to a group represented by the general formula hydrocarbylC(O)O—, preferably alkylC(O)O—.

The term “alkoxy” refers to an alkyl group, preferably a lower alkyl group, having an oxygen attached thereto. Representative alkoxy groups include methoxy, ethoxy, propoxy, tert-butoxy and the like.

The term “alkoxyalkyl” refers to an alkyl group substituted with an alkoxy group and may be represented by the general formula alkyl-O-alkyl.

The term “alkenyl”, as used herein, refers to an aliphatic group containing at least one double bond and is intended to include both “unsubstituted alkenyls” and “substituted alkenyls” the latter of which refers to alkenyl moieties having substituents replacing a hydrogen on one or more carbons of the alkenyl group. Such substituents may occur on one or more carbons that are included or not included in one or more double bonds. Moreover, such substituents include all those contemplated for alkyl groups, as discussed below, except where stability is prohibitive. For example, substitution of alkenyl groups by one or more alkyl, carbocyclyl, aryl, heterocyclyl, or heteroaryl groups is contemplated.

An “alkyl” group or “alkane” is a straight chained or branched non-aromatic hydrocarbon which is completely saturated. Typically, a straight chained or branched alkyl group has from 1 to about 20 carbon atoms, preferably from 1 to about 10 unless otherwise defined. Examples of straight chained and branched alkyl groups include methyl, ethyl, n-propyl, iso-propyl, n-butyl, sec-butyl, tert-butyl, pentyl, hexyl, pentyl and octyl. A C₁-C₆ straight chained or branched alkyl group is also referred to as a “lower alkyl” group.

Moreover, the term “alkyl” (or “lower alkyl”) as used throughout the specification, examples, and claims is intended to include both “unsubstituted alkyls” and “substituted alkyls”, the latter of which refers to alkyl moieties having substituents replacing a hydrogen on one or more carbons of the hydrocarbon backbone. Such substituents, if not otherwise specified, can include, for example, a halogen, a hydroxyl, a carbonyl (such as a carboxyl, an alkoxycarbonyl, a formyl, or an acyl), a thiocarbonyl (such as a thioester, a thioacetate, or a thioformate), an alkoxyl, a phosphoryl, a phosphate, a phosphonate, a phosphinate, an amino, an amido, an amidine, an imine, a cyano, a nitro, an azido, a sulfhydryl, an alkylthio, a sulfate, a sulfonate, a sulfamoyl, a sulfonamido, a sulfonyl, a heterocyclyl, an aralkyl, or an aromatic or heteroaromatic moiety. It will be understood by those skilled in the art that the moieties substituted on the hydrocarbon chain can themselves be substituted, if appropriate. For instance, the substituents of a substituted alkyl may include substituted and unsubstituted forms of amino, azido, imino, amido, phosphoryl (including phosphonate and phosphinate), sulfonyl (including sulfate, sulfonamido, sulfamoyl and sulfonate), and silyl groups, as well as ethers, alkylthios, carbonyls (including ketones, aldehydes, carboxylates, and esters). —CF.sub.3, —CN and the like. Exemplary substituted alkyls are described below. Cycloalkyls can be further substituted with alkyls, alkenyls, alkoxys, alkylthios, aminoalkyls, carbonyl-substituted alkyls, —CF₃, —CN, and the like.

The term “alkylamino”, as used herein, refers to an amino group substituted with at least one alkyl group.

The term “alkylthio”, as used herein, refers to a thiol group substituted with an alkyl group and may be represented by the general formula alkylS—.

The term “alkynyl”, as used herein, refers to an aliphatic group containing at least one triple bond and is intended to include both “unsubstituted alkynyls” and “substituted alkynyls”, the latter of which refers to alkynyl moieties having substituents replacing a hydrogen on one or more carbons of the alkynyl group. Such substituents may occur on one or more carbons that are included or not included in one or more triple bonds. Moreover, such substituents include all those contemplated for alkyl groups, as discussed above, except where stability is prohibitive. For example, substitution of alkynyl groups by one or more alkyl, carbocyclyl, aryl, heterocyclyl, or heteroaryl groups is contemplated.

The terms “amine” and “amino” are art-recognized and refer to both unsubstituted and substituted amines and salts thereof.

The term “aminoalkyl”, as used herein, refers to an alkyl group substituted with an amino group.

The term “aralkyl”, as used herein, refers to an alkyl group substituted with an aryl group.

The term “aryl” as used herein include substituted or unsubstituted single-ring aromatic groups in which each atom of the ring is carbon. Preferably the ring is a 5- to 7-membered ring, more preferably a 6-membered ring. The term “aryl” also includes polycyclic ring systems having two or more cyclic rings in which two or more carbons are common to two adjoining rings wherein at least one of the rings is aromatic, e.g., the other cyclic rings can be cycloalkyls, cycloalkenyls, cycloalkynyls, aryls, heteroaryls, and/or heterocycles. Aryl groups include benzene, naphthalene, phenanthrene, phenol, aniline, and the like.

The terms “carbocycle”, and “carbocyclic”, as used herein, refers to a saturated or unsaturated ring in which each atom of the ring is carbon. The term carbocycle includes both aromatic carbocycles and non-aromatic carbocycles. Non-aromatic carbocycles include both cycloalkane rings, in which all carbon atoms are saturated, and cycloalkene rings, which contain at least one double bond. “Carbocycle” includes 5-7 membered monocyclic and 8-12 membered bicyclic rings. Each ring of a bicyclic carbocycle may be selected from saturated, unsaturated and aromatic rings. Carbocycle includes bicyclic molecules in which one, two or three or more atoms are shared between the two rings. The term “fused carbocycle” refers to a bicyclic carbocycle in which each of the rings shares two adjacent atoms with the other ring. Each ring of a fused carbocycle may be selected from saturated, unsaturated and aromatic rings. In an exemplary embodiment, an aromatic ring, e.g., phenyl, may be fused to a saturated or unsaturated ring, e.g., cyclohexane, cyclopentane, or cyclohexene. Any combination of saturated, unsaturated and aromatic bicyclic rings, as valence permits, is included in the definition of carbocyclic. Exemplary “carbocycles” include cyclopentane, cyclohexane, bicyclo[2.2.1]heptane, 1,5-cyclooctadiene, 1,2,3,4-tetrahydronaphthalene, bicyclo[4.2.0]oct-3-ene, naphthalene and adamantane. Exemplary fused carbocycles include decalin, naphthalene, 1,2,3,4-tetrahvdronaphthalene, bicyclo[4.2.0]octane, 4,5,6,7-tetrahydro-1H-indene and bicyclo[4.1.0]hept-3-ene. “Carbocycles” may be substituted at any one or more positions capable of bearing a hydrogen atom.

A “cycloalkyl” group is a cyclic hydrocarbon which is completely saturated. “Cycloalkyl” includes monocyclic and bicyclic rings. Typically, a monocyclic cycloalkyl group has from 3 to about 10 carbon atoms, more typically 3 to 8 carbon atoms unless otherwise defined. The second ring of a bicyclic cycloalkyl may be selected from saturated, unsaturated and aromatic rings. Cycloalkyl includes bicyclic molecules in which one, two or three or more atoms are shared between the two rings. The term “fused cycloalkyl” refers to a bicyclic cycloalkyl in which each of the rings shares two adjacent atoms with the other ring. The second ring of a fused bicyclic cycloalkyl may be selected from saturated, unsaturated and aromatic rings. A “cycloalkenyl” group is a cyclic hydrocarbon containing one or more double bonds.

The term “carbocyclylalkyl”, as used herein, refers to an alkyl group substituted with a carbocycle group.

The term “carbonate” is art-recognized and refers to a group —OCO₂—R₁₀, wherein R₁₀ represents a hydrocarbyl group.

The term “carboxy”, as used herein, refers to a group represented by the formula —CO₂H.

The term “ester”, as used herein, refers to a group —C(O)OR₁₀ wherein R₁₀ represents a hydrocarbyl group.

The term “ether”, as used herein, refers to a hydrocarbyl group linked through an oxygen to another hydrocarbyl group. Accordingly, an ether substituent of a hydrocarbyl group may be hydrocarbyl-O—. Ethers may be either symmetrical or unsymmetrical. Examples of ethers include, but are not limited to, heterocycle-O-heterocycle and aryl-O-heterocycle. Ethers include “alkoxyalkyl” groups, which may be represented by the general formula alkyl-O-alkyl.

The terms “halo” and “halogen” as used herein means halogen and includes chloro, fluoro, bromo, and iodo.

The term “heteroaralkyl”, as used herein, refers to an alkyl group substituted with a hetaryl group.

The term “heteroalkyl”, as used herein, refers to a saturated or unsaturated chain of carbon atoms and at least one heteroatom, wherein no two heteroatoms are adjacent.

The terms “heteroaryl” and “hetaryl” include substituted or unsubstituted aromatic single ring structures, preferably 5- to 7-membered rings, more preferably 5- to 6-membered rings, whose ring structures include at least one heteroatom, preferably one to four heteroatoms, more preferably one or two heteroatoms. The terms “heteroaryl” and “hetaryl” also include polycyclic ring systems having two or more cyclic rings in which two or more carbons are common to two adjoining rings wherein at least one of the rings is heteroaromatic, e.g., the other cyclic rings can be cycloalkyls, cycloalkenyls, cycloalkynyls, aryls, heteroaryls, and/or heterocyclyls. Heteroaryl groups include, for example, pyrrole, furan, thiophene, imidazole, oxazole, thiazole, pyrazole, pyridine, pyrazine, pyridazine, and pyrimidine, and the like.

The term “heteroatom” as used herein means an atom of any element other than carbon or hydrogen. Preferred heteroatoms are nitrogen, oxygen, and sulfur.

The terms “heterocyclyl”, “heterocycle”, and “heterocyclic” refer to substituted or unsubstituted non-aromatic ring structures, preferably 3- to 10-membered rings, more preferably 3- to 7-membered rings, whose ring structures include at least one heteroatom, preferably one to four heteroatoms, more preferably one or two heteroatoms. The terms “heterocyclyl” and “heterocyclic” also include polycyclic ring systems having two or more cyclic rings in which two or more carbons are common to two adjoining rings wherein at least one of the rings is heterocyclic, e.g., the other cyclic rings can be cycloalkyls, cycloalkenyls, cycloalkynyls, aryls, heteroaryls, and/or heterocyclyls. Heterocyclyl groups include, for example, piperidine, piperazine, pyrrolidine, morpholine, lactones, lactams, and the like.

The term “heterocyclylalkyl”, as used herein, refers to an alkyl group substituted with a heterocycle group.

The term “hydrocarbyl”, as used herein, refers to a group that is bonded through a carbon atom that does not have a ═O or ═S substituent, and typically has at least one carbon-hydrogen bond and a primarily carbon backbone, but may optionally include heteroatoms. Thus, groups like methyl, ethoxyethyl, 2-pyridyl, and trifluoromethyl are considered to be hydrocarbyl for the purposes of this application, but substituents such as acetyl (which has a ═O substituent on the linking carbon) and ethoxy (which is linked through oxygen, not carbon) are not. Hydrocarbyl groups include, but are not limited to aryl, heteroaryl, carbocycle, heterocyclyl, alkyl, alkenyl, alkynyl, and combinations thereof.

The term “hydroxyalkyl”, as used herein, refers to an alkyl group substituted with a hydroxy group.

The term “lower” when used in conjunction with a chemical moiety, such as, acyl, acyloxy, alkyl, alkenyl, alkynyl, or alkoxy is meant to include groups where there are ten or fewer non-hydrogen atoms in the substituent, preferably six or fewer. A “lower alkyl”, for example, refers to an alkyl group that contains ten or fewer carbon atoms, preferably six or fewer. In certain embodiments, acyl, acyloxy, alkyl, alkenyl, alkynyl, or alkoxy substituents defined herein are respectively lower acyl, lower acyloxy, lower alkyl, lower alkenyl, lower alkynyl, or lower alkoxy, whether they appear alone or in combination with other substituents, such as in the recitations hydroxyalkyl and aralkyl (in which case, for example, the atoms within the aryl group are not counted when counting the carbon atoms in the alkyl substituent).

The terms “polycyclyl”, “polycycle”, and “polycyclic” refer to two or more rings (e.g., cycloalkyls, cycloalkenyls, cycloalkynyls, aryls, heteroaryls, and/or heterocyclyls) in which two or more atoms are common to two adjoining rings, e.g., the rings are “fused rings”. Each of the rings of the polycycle can be substituted or unsubstituted. In certain embodiments, each ring of the polycycle contains from 3 to 10 atoms in the ring, preferably from 5 to 7.

The term “silyl” refers to a silicon moiety with three hydrocarbyl moieties attached thereto.

The term “substituted” refers to moieties having substituents replacing a hydrogen on one or more carbons of the backbone. It will be understood that “substitution” or “substituted with” includes the implicit proviso that such substitution is in accordance with permitted valence of the substituted atom and the substituent, and that the substitution results in a stable compound, e.g., which does not spontaneously undergo transformation such as by rearrangement, cyclization, elimination, etc. As used herein, the term “substituted” is contemplated to include all permissible substituents of organic compounds. In a broad aspect, the permissible substituents include acyclic and cyclic, branched and unbranched, carbocyclic and heterocyclic, aromatic and non-aromatic substituents of organic compounds. The permissible substituents can be one or more and the same or different for appropriate organic compounds. For purposes of this invention, the heteroatoms such as nitrogen may have hydrogen substituents and/or any permissible substituents of organic compounds described herein which satisfy the valences of the heteroatoms. Substituents can include any substituents described herein, for example, a halogen, a hydroxyl, a carbonyl (such as a carboxyl, an alkoxycarbonyl, a formyl, or an acyl), a thiocarbonyl (such as a thioester, a thioacetate, or a thioformate), an alkoxyl, a phosphoryl, a phosphate, a phosphonate, a phosphinate, an amino, an amido, an amidine, an imine, a cyano, a nitro, an azido, a sulfhydryl, an alkylthio, a sulfate, a sulfonate, a sulfamoyl, a sulfonamido, a sulfonyl, a heterocyclyl, an aralkyl, or an aromatic or heteroaromatic moiety. It will be understood by those skilled in the art that substituents can themselves be substituted, if appropriate. Unless specifically stated as “unsubstituted,” references to chemical moieties herein are understood to include substituted variants. For example, reference to an “aryl” group or moiety implicitly includes both substituted and unsubstituted variants.

The term “sulfate” is art-recognized and refers to the group —OSO.sub.3H, or a pharmaceutically acceptable salt thereof.

The term “sulfoxide” is art-recognized and refers to the group —S(O)—R₁₀ wherein R₁₀ represents a hydrocarbyl.

The term “sulfonate” is art-recognized and refers to the group SO₃H, or a pharmaceutically acceptable salt thereof.

The term “protecting group” refers to a group of atoms that, when attached to a reactive functional group in a molecule, mask, reduce or prevent the reactivity of the functional group. Typically, a protecting group may be selectively removed as desired during the course of a synthesis. Examples of protecting groups can be found in Greene and Wuts, Protective Groups in Organic Chemistry, 3.sup.rd Ed., 1999, John Wiley & Sons, NY and Harrison et al., Compendium of Synthetic Organic Methods, Vols. 1-8, 1971-1996. John Wiley & Sons, NY. Representative nitrogen protecting groups include, but are not limited to, formyl, acetyl, trifluoroacetyl, benzyl, benzyloxycarbonyl (“CBZ”), tert-butoxycarbonyl (“Boc”), trimethylsilyl (“TMS”), 2-trimethylsilyl-ethanesulfonyl (“TES”), trityl and substituted trityl groups, allyloxycarbonyl, 9-fluorenylmethyloxycarbonyl (“FMOC”), nitro-veratryloxycarbonyl (“NVOC”) and the like. Representative hydroxylprotecting groups include, but are not limited to, those where the hydroxyl group is either acylated (esterified) or alkylated such as benzyl and trityl ethers, as well as alkyl ethers, tetrahydropyranyl ethers, trialkylsilyl ethers (e.g., TMS or TIPS groups), glycol ethers, such as ethylene glycol and propylene glycol derivatives and allyl ethers.

The term “healthcare providers” refers to individuals or organizations that provide healthcare services to a person, community, etc. Examples of “healthcare providers” include doctors, hospitals, continuing care retirement communities, skilled nursing facilities, subacute care facilities, clinics, multispecialty clinics, freestanding ambulatory centers, home health agencies, and HMO's.

As used herein, a therapeutic that “prevents” a disorder or condition refers to a compound that, in a statistical sample, reduces the occurrence of the disorder or condition in the treated sample relative to an untreated control sample, or delays the onset or reduces the severity of one or more symptoms of the disorder or condition relative to the untreated control sample.

The term “treating” includes prophylactic and/or therapeutic treatments. The term “prophylactic or therapeutic” treatment is art-recognized and includes administration to the host of one or more of the subject compositions. If it is administered prior to clinical manifestation of the unwanted condition (e.g., disease or other unwanted state of the host animal) then the treatment is prophylactic (i.e., it protects the host against developing the unwanted condition), whereas if it is administered after manifestation of the unwanted condition, the treatment is therapeutic, (i.e., it is intended to diminish, ameliorate, or stabilize the existing unwanted condition or side effects thereof).

The term “prodrug” is intended to encompass compounds which, under physiologic conditions, are converted into the therapeutically active agents of the present invention (e.g., a compound of formula I). A common method for making a prodrug is to include one or more selected moieties which are hydrolyzed under physiologic conditions to reveal the desired molecule. In other embodiments, the prodrug is converted by an enzymatic activity of the host animal. For example, esters or carbonates (e.g., esters or carbonates of alcohols or carboxylic acids) are preferred prodrugs of the present invention. In certain embodiments, some or all of the compounds of formula I in a formulation represented above can be replaced with the corresponding suitable prodrug, e.g., wherein a hydroxyl in the parent compound is presented as an ester or a carbonate or carboxylic acid present in the parent compound is presented as an ester.

The term “masking moiety” as used herein, refers to the chemical moiety that is a covalently bound modification of a pharmacophore that renders the compounds of the present invention to which it is attached prodrugs. A masking moiety is cleavable under, for example, acidic conditions, basic conditions, or physiologic conditions. When the masking moiety is cleaved, the prodrugs are converted to the therapeutically active agents of the present invention. Esters and carbonates can be used to mask hydroxyls, carbamates and amides can be used to mask amines, carboxyls can be masked as esters, etc., and in certain embodiments the precise masking moiety can be selected to be cleaved under conditions particular to a region of the digestive tract. For example, an amine or hydroxyl can be acylated by a 4-aminobutanoyl group, to form a prodrug that can be administered as a salt of the amine. In the acidic conditions of the stomach, the amino group will remain protonated, masking its nucleophilicity. In the more basic conditions of the small intestines, the ammonium group will be deprotonated, revealing the nucleophilic amine, which can nucleophilicly attack the amide or ester formed by the butanoyl group, ultimately revealing the amide or ester with the concomitant release of the protecting group as a lactam.

Robustness in pharmacophore design can be increased by using diverse ensembles of classifiers, while self-assessment of classifiers can be obtained by defining an appropriate uncertainty measure by using probabilistic or fuzzy classifiers, or by using the degree of agreement of an ensemble of classifiers (this decision is more confident if most the individual classifiers agree on a specific value or label of a potential inhibitor compound). When combining multiple decisions, the uncertainty can be used as a weight in the fusion. In multimodal data fusion of physicochemical properties, it is preferable to incorporate unlabeled data, a less expensive and time-consuming procedure, in order to improve a statistical learning process or to support an interactive labeling process. Integration of unlabeled data in a supervised machine learning procedure can be done by semi-supervised learning and active learning. A classification architecture designed for a real-world application needs to handle missing data by temporal smoothing of the results. In the same study, classification architecture has been proposed that classified individual channels of physicochemical properties separately using an uncertainty measure, while prevented false classifications by applying a sample reject mechanism. Missing values were constructed by the integration of subsequent temporal integration. In order to design the CoV2 Spike GP and human ACE2 interaction interface specific 3D pharmacophore model described herein a holistic model encompassing all unique features of each potential must be developed.

Energy minimizations were used to remove any residual geometrical strain in each molecular system with CoV2 Spike GP, ACE2 with or without any compounds using the Charmm27 force field as it is implemented into the Gromacs suite, version 4.5.5. Molecular systems were then subjected to unrestrained Molecular Dynamics Simulations (MDS) using the Gromacs suite, version 4.5.5. MDS took place in a SPC water-solvated, periodic environment. Water molecules were added using the truncated octahedron box extending 7 Å from each atom. Molecular systems were neutralized with counter-ions as required. For the purposes of this study all MDS were performed using the NVT ensemble in a canonical environment, at 300 K and a step size equal to 2 femtoseconds for a total 100 nanoseconds simulation time.

In order to confirm in silico the most promising complex structures of CoV2 Spike GP and ACE2 with the top hit compounds as proposed by the pharmacophore model, the docking suite ZDOCK was used. Docking experiments were conducted on the models that had been energetically minimized and conformationally optimized using molecular dynamics simulations. ZDOCK uses a scoring function that returns electrostatic, hydrophobic and desolvation energies as well as performing a fast pairwise shape complementarity evaluation.

For the construction of the 3D pharmacophore model more than 435 physicochemical descriptors were applied to the analysis of both the CoV2 Spike GP and human ACE2 proteins as well as the dataset of inhibiting compounds. The aim of this analysis is to achieve maximal compatibility and complementarity between each protein site and each screened inhibitor compound.

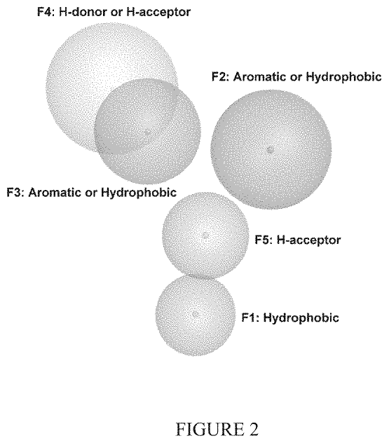

An exemplary (reduced) pharmacophore model is depicted in FIG. 2. It was determined that a set of criteria had to be met, in order for a potential inhibitor compound to be in silico annotated as potentially active. Firstly, there should be a hydrophobic group (FIG. 2: F1); an aromatic or hydrophobic group (FIG. 2: F2); an aromatic or hydrophobic group (FIG. 2: F3); an H-donor or H-acceptor group (FIG. 2: F4) and an H-acceptor group (FIG. 2: F5).

The proposed, tailor-made for CoV2 Spike GP and human ACE2 proteins, 3D pharmacophore model described herein, provides the means for rapid, high-throughput virtual screening of potential anti-CoV2 modulators thus facilitating, optimizing and speeding up the search for the discovery of a potent anti-COVID19 agent.

In one aspect, the invention provides a compound weighing less than about 10,000 Daltons (preferably less than about 7500 Da, less than about 5000 Da, less than about 3000 Da, or even less than about 2000 Da), comprising a scaffold having a plurality of branches (preferably at least 3, or 4, 5, or more branches), wherein some or all of the branches (preferably at least 3, or 4, 5, or more branches) terminate in a pharmacophore having an affinity for a biological target particularly anti-CoV2 modulators, or a pharmaceutically acceptable salt thereof.

In certain embodiments, the compound is dendrimer, e.g., where the branches are identical or similar (e.g., differing primarily in the terminal pharmacophore).

In certain embodiments, the scaffold is a molecular framework for joining the various branches and pharmacophores, and may be oligomeric, dendritic, cyclic, acyclic, branched, linear, or any other suitable form for covalently linking the various elements of the compound together. Similarly, the branches may be long or short, cyclic or acyclic, or any other suitable configuration for covalently linking the pharmacophores to the scaffold. The primary function of the scaffold and branches is to present the pharmacophores in an environment that is sterically unhindered enough for the pharmacophore to interact with its target receptor. Thus, a compact scaffold (e.g., where branches can be coupled to neighboring hydroxyl groups) may be paired with longer branches to avoid steric crowding of the pharmacophores, while a scaffold with greater spacing between branching points (e.g., an oligomeric chain) may be able to utilize shorter branches without compromising interaction of the pharmacophores with their target receptors. In certain embodiments, each branch terminates in a pharmacophore. In certain embodiments, at least one branch does not terminate in a pharmacophore.

The pharmacophores are chemical moieties capable of interacting with (e.g., activating or inactivating, agonizing or antagonizing) a biological receptor, preferably on a selective basis. In some embodiments, a pharmacophore is essentially a drug molecule that is covalently attached to the rest of the compound; that is, a pharmacophore, severed from its branch and scaffold, retains its ability to interact with the receptor that it targets as part of the compound and has biological activity of its own.

For any given molecule of the compound, the pharmacophores can be identical for all branches that terminate in a pharmacophore, or some branches may terminate in one pharmacophore while other branches terminate in a different pharmacophore. In some embodiments, each branch terminates in a different pharmacophore. Different pharmacophores of a molecule may target the same receptor or different receptors. In certain embodiments, pharmacophores are displayed on the scaffold in an arrangement that permits two or more pharmacophores of a compound to simultaneously interact with two or more target receptors on the surface of a single cell. When a compound comprises at least two different pharmacophores, administering the compound may provide an additive therapeutic effect (or even a synergistic therapeutic effect) relative to the therapeutic effect of administering a mixture of analogous compounds where each pharmacophore of a compound is the same, but the mixture provides pharmacophores in the same ratio present in the multi-pharmacophore compound (in other words, as though the pharmacophores of the multi-pharmacophore compound were redistributed among scaffolds such that the pharmacophores of a single scaffold were all identical, but the mixture of scaffolds comprises the same pharmacophores in corresponding proportions and amounts as in the multi-pharmacophore compound).

In certain embodiments, one or more pharmacophores have an affinity for a biological target. Such biological targets include, but are not limited to receptors, ion channels, and transporters. In certain embodiments, the receptor is a PRR-type receptor (Pattern Recognition Receptor), such as a TLR-type receptor. In certain embodiments, one or more pharmacophores have an affinity for a bile acid receptor, taste receptor, olfactory receptor, TGR-type, or GPR-type receptor.

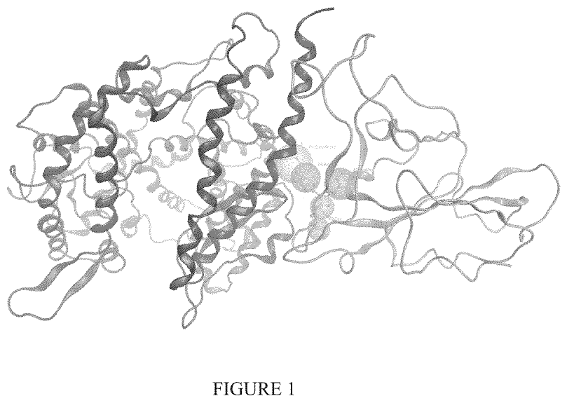

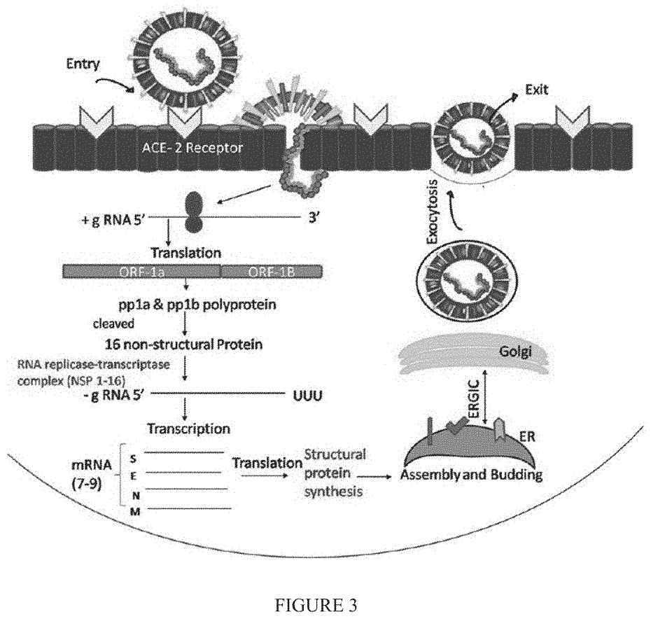

Particular embodiments of the compounds of the invention are described herein and in FIGS. 1-3. In certain embodiments, the pharmacophores that are incorporated into the compounds of the invention are derived from bioactive drug compounds. In certain embodiments, the pharmacophore moieties are related to the parent structure of the bioactive drug compounds by chemical modification (e.g., substitution) of the parent structure. In example embodiments, a parent structure is to be modified by a linking moiety that effectively attaches the pharmacophore to a scaffold, as described herein.

Modifications of bioactive drug compound include substitution, truncation, stereocenter inversion, isomerization, or hybridization change. In certain embodiments, a bioactive drug compound is substituted at any substitutable position, including a heteroatom (e.g., O, N, S, Sc, P), a hydrogen-bearing sp²-hybridized carbon, or a hydrogen-bearing sp³-hybridized CH.

A person of ordinary skill in the art would appreciate that a bioactive drug compound can be incorporated as a pharmacophore into the compounds of the invention in any one of a variety of spatial orientations, and at any one of a variety of substitutable positions, as described herein. A person of ordinary skill in the art can readily synthesize a number of such compounds in order to determine which orientations and connectivities exhibit acceptable biological activity for a given application. In example embodiments, a compound with a pharmacophore having appropriate orientation and connectivity retains the type of biological activity of the parent bioactive drug compound, though the activity of the compound bearing the pharmacophore may be stronger or weaker than that of the parent bioactive drug compound. In certain embodiments, a suitable orientation and connectivity is determined when the pharmacophore exhibits at least 99%, 98%, 95%, 90%, 85%, 80%, 75% 70%, 65%, 60%, 55%, or 50% of the biological activity of parent compound.

A pharmacophore model can be constructed from structural information of biomolecule components playing a role in activation including definition of atom position. Small molecules with complementary features to components of the target biomolecule, such as a component playing a role in activation, have the potential to interfere with configuration changes and/or stabilization necessary for activation and thus have therapeutic utility.

In various embodiments, in silico approaches can be used for de novo structure design with a fragment-based approach employing contact statistics, 3D surface models, and docked ligands as templates. From the spatial position information, and/or from other parameters described above, one can derive 3D ligand-receptor models (e.g., interaction pattern, pharmacophore schemes), surface maps (e.g., topography/shape, electrostatic profile, hydrophobicity, protein flexibility), and docking models (e.g., scoring system for ligand binding, minimum energy calculation).

A pharmacophore model or scheme is generally a set of structural features in a ligand that are related, preferably directly related, to the ligand's recognition at a receptor site and its biological activity. Pharmacophore features can be derived from corresponding donor, acceptor, aromatic, hydrophobic, and/or acidic or basic moieties of the corresponding target biomolecule, especially those features on domains participating in dimerization and activation mechanisms. It shall be understood that additional information about the nature of the atoms in the target biomolecule being used in a pharmacophore scheme, and not simply the spatial location of the atoms, can assist in the modelling process of a new chemical lead. These characteristics include, but are not limited to, the pKa values of the atoms, the rotational rigidity of the bonds holding the atoms in place, the nature of the bonds themselves (single, double, resonant, or otherwise), the projected directionality of hydrogen bond donors and acceptors, etc.

Typical feature components useful in generating a pharmacophore scheme include, but are not limited to, atomic position: atomic radii; hydrogen bond donor features; hydrogen bond acceptor features; aromatic features: donor features; acceptor features: anion features; cation features; acceptor and anion features; donor and cation features: donor and acceptor features; acid and anion features: hydrophobic features, hydrogen bond directionality, and metal ligands. Such features can be located, for example, at a single atom, centroids of atoms, or at a projected directional position in space.

Pharmacophoric structural features can be represented by labeled points in space. Each ligand can be assigned an annotation, which is a set of structural features that may contribute to the ligand's pharmacophore. In various embodiments, a database of annotated ligands can be searched with a query that represents a pharmacophore hypothesis. The result of such a search is a set of matches that align the pharmacophoric features of the query to the pharmacophoric features present in the ligands of the searched database. The number of hits within the database depends, at least in part, upon the size of the database and the restrictiveness of the pharmacophore query (e.g., partial matches, number of features, etc.). Properties and parameters of the molecules present within the search database are used to focus the outcome of the query. For example, compounds with a defined range of molecular weight (MW) or lipophilicity (log P) can be present in the searched section of the library database of compounds.

The subject methods find use in the screening of a variety of different candidate molecules (e.g., potentially therapeutic candidate molecules). As described above, candidate molecules can be searched using a pharmacophore query. Candidate molecules encompass numerous chemical classes, though typically they are organic molecules, preferably small organic compounds having a molecular weight of more than 50 and less than about 2,500 Daltons. Candidate molecules can comprise functional groups for structural interaction with proteins, particularly hydrogen bonding, and can include at least an amine, carbonyl, hydroxyl or carboxyl group, preferably at least two of the functional chemical groups. The candidate molecules can comprise cyclical carbon or heterocyclic structures and/or aromatic or polyaromatic structures substituted with one or more of the above functional groups.

In preferred embodiments, the candidate molecules are compounds in a library database of compounds. One of skill in the art will be generally familiar with, for example, numerous databases for commercially available compounds for screening (see e.g., ZINC database, UCSF, with 2.7 million compounds over 12 distinct subsets of molecules; Irwin and Shoichet (2005) J Chem Inf Model 45, 177-182). One of skill in the art will also be familiar with a variety of search engines to identify commercial sources or desirable compounds and classes of compounds for further testing (see e.g., ZINC database; eMolecules.com; and electronic libraries of commercial compounds provided by vendors, for example: ChemBridge, Princeton BioMolecular, Ambinter SARL, Enamine, ASDI, Life Chemicals etc).

Candidate molecules for screening according to the methods described herein include both lead-like compounds and drug-like compounds. A lead-like compound is generally understood to have a relatively smaller scaffold-like structure (e.g., molecular weight of about 150 to about 350 D) with relatively fewer features (e.g., less than about 3 hydrogen donors and/or less than about 6 hydrogen acceptors; hydrophobicity character×log P of about −2 to about 4) (see e.g., Angewante (1999) Chemie Int. ed. Engl. 24, 3943-3948). In contrast, a drug-like compound is generally understood to have a relatively larger scaffold (e.g., molecular weight of about 150 to about 500 D) with relatively more numerous features (e.g., less than about 10 hydrogen acceptors and/or less than about 8 rotatable bonds: hydrophobicity character×log P of less than about 5) (see e.g., Lipinski (2000) J. Pharm. Tox. Methods 44, 235-249). Preferably, initial screening is performed with lead-like compounds.

When designing a lead from spatial orientation data, it can be useful to understand that certain molecular structures are characterized as being “drug-like”. Such characterization can be based on a set of empirically recognized qualities derived by comparing similarities across the breadth of known drugs within the pharmacopoeia. While it is not required for drugs to meet all, or even any, of these characterizations, it is far more likely for a drug candidate to meet with clinical success if it is drug-like.

Several of these “drug-like” characteristics have been summarized into the four rules of Lipinski (generally known as the “rules of fives” because of the prevalence of the number 5 among them). While these rules generally relate to oral absorption and are used to predict bioavailability of compound during lead optimization, they can serve as effective guidelines for constructing a lead molecule during rational drug design efforts such as may be accomplished by using the methods of the present invention.

The four “rules of five” state that a candidate drug-like compound should have at least three of the following characteristics: (i) a weight less than 500 Daltons: (ii) a log of P less than 5; (iii) no more than 5 hydrogen bond donors (expressed as the sum of OH and NH groups); and (iv) no more than 10 hydrogen bond acceptors (the sum of N and O atoms). Also, drug-like molecules typically have a span (breadth) of between about 8.ANG. to about 15.ANG. It will be understood that a candidate molecule, or even a selected molecule, may not meet all, or even any, of these characterizations. Nonetheless, the above guidelines are helpful in drug screening and design.

Candidate molecules identified as being complementary to certain features of a target biomolecule as compared to a pharmacophore model (e.g., through a pharmacophore query as described above) can be further selected according to docking affinity for the target biomolecule. In addition to pharmacophore model generation for database queries, a second sequential and complementary method for compound identification and design can be employed. Pharmacophore queries can filter out compounds quickly and docking and scoring can evaluate ligand-target biomolecule binding more accurately. In the case of protein or enzyme target biomolecules, amino acid residues of different domains in an inactive conformation can be used to define the docking site.

Candidate molecules selected according to pharmacophore query and/or further selected according to docking analysis can be tested for effect on the target biomolecule. Assessment of effect of a molecule on biomolecule function (e.g., inhibition of enzymatic activity) can be assessed by various methods known in the art (see e.g., Examples 1-3). For example, inhibitory effect of a candidate molecule on the catalytic activity of a target enzyme can be assessed by known activity assays specific for the target enzyme (see e.g., Reymond, ed. (2006) Enzyme Assays: High-throughput Screening, Genetic Selection and Fingerprinting, John Wiley & Sons, 386 p., ISBN-10: 3527310959; Eisenthall and Danson, Ed. (2002) Enzyme Assays, 2d edition, Oxford University Press, 384 p., ISBN-10: 0199638209). As described herein, an in-cell Western (ICW) screening protocol can be used to evaluate candidate compounds (see e.g., Example 1; Chen et al. (2005) Analytical Biochemistry 338, 136-142). Also as described herein, a MTT Cell Proliferation Assay can be used to evaluate candidate compounds (see e.g., Example 2). Also as described herein, an EGF inhibitor assay can be used to evaluate candidate compounds (see e.g., Example 3; Mukku (1984) J. Biol. Chem. 259, 6543-6546; Duh et al. (1990) World J. Surgery 14, 410-418; Lokeshwar et al. (1989) J. Biol. Chem. 264(32), 19318-19326).

Further refinement of candidate molecules can be conducted. For example, data from biological assays can be correlated with the docking model so as to further refine lead-like molecules and/or drug-like molecules. Various software packages (e.g., MOE) can be employed to visualize active compound interaction with a target biomolecule to identify sites on the template suitable for modification by de novo design. Analogs of active compounds can be identified using similarity and sub-structure searches (see e.g., SciFinder: eModel). Available analogs can be analyzed according to docking and scoring procedures described above. Analogs with desirable docking scores can be acquired and further tested for biological effect on the target biomolecule according to methods described herein. One skilled in the art will understand these, and other, methods of refining and further developing candidate molecules identified by the methods presented herein.

The compositions and methods of the present invention may be utilized to treat an individual in need thereof. In certain embodiments, the individual is a mammal such as a human, or a non-human mammal. When administered to an animal, such as a human, the composition or the compound is preferably administered as a pharmaceutical composition comprising, for example, a compound of the invention and a pharmaceutically acceptable carrier. Pharmaceutically acceptable carriers are well known in the art and include, for example, aqueous solutions such as water or physiologically buffered saline or other solvents or vehicles such as glycols, glycerol, oils such as olive oil, or injectable organic esters. In a preferred embodiment, when such pharmaceutical compositions are for human administration, particularly for invasive mutes of administration (i.e., routes, such as injection or implantation, that circumvent transport or diffusion through an epithelial barrier), the aqueous solution is pyrogen-free, or substantially pyrogen-free. The excipients can be chosen, for example, to effect delayed release of an agent or to selectively target one or more cells, tissues or organs. The pharmaceutical composition can be in dosage unit form such as tablet, capsule (including sprinkle capsule and gelatin capsule), granule, lyophile for reconstitution, powder, solution, syrup, suppository, injection or the like. The composition can also be present in a transdermal delivery system, e.g., a skin patch. The composition can also be present in a solution suitable for topical administration, such as an eye drop.

A pharmaceutically acceptable carrier can contain physiologically acceptable agents that act, for example, to stabilize, increase solubility or to increase the absorption of a compound such as a compound of the invention. Such physiologically acceptable agents include, for example, carbohydrates, such as glucose, sucrose or dextrans, antioxidants, such as ascorbic acid or glutathione, chelating agents, low molecular weight proteins or other stabilizers or excipients. The choice of a pharmaceutically acceptable carrier, including a physiologically acceptable agent, depends, for example, on the route of administration of the composition. The preparation or pharmaceutical composition can be a self-emulsifying drug delivery system or a self-microemulsifying drug delivery system. The pharmaceutical composition (preparation) also can be a liposome or other polymer matrix, which can have incorporated therein, for example, a compound of the invention. Liposomes, for example, which comprise phospholipids or other lipids, are nontoxic, physiologically acceptable and metabolizable carriers that are relatively simple to make and administer.

The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

The phrase “pharmaceutically acceptable carrier” as used herein means a pharmaceutically acceptable material, composition or vehicle, such as a liquid or solid filler, diluent, excipient, solvent or encapsulating material. Each carrier must be “acceptable” in the sense of being compatible with the other ingredients of the formulation and not injurious to the patient. Some examples of materials which can serve as pharmaceutically acceptable carriers include: (1) sugars, such as lactose, glucose and sucrose; (2) starches, such as corn starch and potato starch; (3) cellulose, and its derivatives, such as sodium carboxymethyl cellulose, ethyl cellulose and cellulose acetate; (4) powdered tragacanth; (5) malt; (6) gelatin; (7) talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol; (12) esters, such as ethyl oleate and ethyl laurate: (13) agar; (14) buffering agents, such as magnesium hydroxide and aluminum hydroxide; (15) alginic acid; (16) pyrogen-free water: (17) isotonic saline; (18) Ringer's solution; (19) ethyl alcohol; (20) phosphate buffer solutions; and (21) other non-toxic compatible substances employed in pharmaceutical formulations.

A pharmaceutical composition (preparation) can be administered to a subject by any of a number of routes of administration including, for example, orally (for example, drenches as in aqueous or non-aqueous solutions or suspensions, tablets, capsules (including sprinkle capsules and gelatin capsules), boluses, powders, granules, pastes for application to the tongue); absorption through the oral mucosa (e.g., sublingually): anally, rectally or vaginally (for example, as a pessary, cream or foam); parenterally (including intramuscularly, intravenously, subcutaneously or intrathecally as, for example, a sterile solution or suspension); nasally; intraperitoneally; subcutaneously; transdermally (for example as a patch applied to the skin); and topically (for example, as a cream, ointment or spray applied to the skin, or as an eye drop). The compound may also be formulated for inhalation. In certain embodiments, a compound may be simply dissolved or suspended in sterile water. Details of appropriate routes of administration and compositions suitable for same can be found in, for example, U.S. Pat. Nos. 6,110,973, 5,763,493, 5,731,000, 5,541,231, 5,427.798, 5,358,970 and 4,172,896, as well as in patents cited therein.

The formulations may conveniently be presented in unit dosage form and may be prepared by any methods well known in the art of pharmacy. The amount of active ingredient which can be combined with a carrier material to produce a single dosage form will vary depending upon the host being treated, the particular mode of administration. The amount of active ingredient that can be combined with a carrier material to produce a single dosage form will generally be that amount of the compound which produces a therapeutic effect. Generally, out of one hundred percent, this amount will range from about 1 percent to about ninety-nine percent of active ingredient, preferably from about 5 percent to about 70 percent, most preferably from about 10 percent to about 30 percent.

Methods of preparing these formulations or compositions include the step of bringing into association an active compound, such as a compound of the invention, with the carrier and, optionally, one or more accessory ingredients. In general, the formulations are prepared by uniformly and intimately bringing into association a compound of the present invention with liquid carriers, or finely divided solid carriers, or both, and then, if necessary, shaping the product.

Formulations of the invention suitable for oral administration may be in the form of capsules (including sprinkle capsules and gelatin capsules), cachets, pills, tablets, lozenges (using a flavored basis, usually sucrose and acacia or tragacanth), lyophile, powders, granules, or as a solution or a suspension in an aqueous or non-aqueous liquid, or as an oil-in-water or water-in-oil liquid emulsion, or as an elixir or syrup, or as pastilles (using an inert base, such as gelatin and glycerin, or sucrose and acacia) and/or as mouth washes and the like, each containing a predetermined amount of a compound of the present invention as an active ingredient. Compositions or compounds may also be administered as a bolus, electuary or paste.

To prepare solid dosage forms for oral administration (capsules (including sprinkle capsules and gelatin capsules), tablets, pills, dragees, powders, granules and the like), the active ingredient is mixed with one or more pharmaceutically acceptable carriers, such as sodium citrate or dicalcium phosphate, and/or any of the following: (1) fillers or extenders, such as starches, lactose, sucrose, glucose, mannitol, and/or silicic acid; (2) binders, such as, for example, carboxymethylcellulose, alginates, gelatin, polyvinyl pyrrolidone, sucrose and/or acacia; (3) humectants, such as glycerol; (4) disintegrating agents, such as agar-agar, calcium carbonate, potato or tapioca starch, alginic acid, certain silicates, and sodium carbonate: (5) solution retarding agents, such as paraffin; (6) absorption accelerators, such as quaternary ammonium compounds; (7) wetting agents, such as, for example, cetyl alcohol and glycerol monostearate; (8) absorbents, such as kaolin and bentonite clay; (9) lubricants, such a talc, calcium stearate, magnesium stearate, solid polyethylene glycols, sodium lauryl sulfate, and mixtures thereof; (10) complexing agents, such as, modified and unmodified cyclodextrins; and (11) coloring agents. In the case of capsules (including sprinkle capsules and gelatin capsules), tablets and pills, the pharmaceutical compositions may also comprise buffering agents. Solid compositions of a similar type may also be employed as fillers in soft and hard-filled gelatin capsules using such excipients as lactose or milk sugars, as well as high molecular weight polyethylene glycols and the like.

A tablet may be made by compression or molding, optionally with one or more accessory ingredients. Compressed tablets may be prepared using binder (for example, gelatin or hydroxypropylmethyl cellulose), lubricant, inert diluent, preservative, disintegrant (for example, sodium starch glycolate or cross-linked sodium carboxymethyl cellulose), surface-active or dispersing agent. Molded tablets may be made by molding in a suitable machine a mixture of the powdered compound moistened with an inert liquid diluent.

The tablets, and other solid dosage forms of the pharmaceutical compositions, such as dragees, capsules (including sprinkle capsules and gelatin capsules), pills and granules, may optionally be scored or prepared with coatings and shells, such as enteric coatings and other coatings well known in the pharmaceutical-formulating art. They may also be formulated so as to provide slow or controlled release of the active ingredient therein using, for example, hydroxypropylmethyl cellulose in varying proportions to provide the desired release profile, other polymer matrices, liposomes and/or microspheres. They may be sterilized by, for example, filtration through a bacteria-retaining filter, or by incorporating sterilizing agents in the form of sterile solid compositions that can be dissolved in sterile water, or some other sterile injectable medium immediately before use. These compositions may also optionally contain opacifying agents and may be of a composition that they release the active ingredient(s) only, or preferentially, in a certain portion of the gastrointestinal tract, optionally, in a delayed manner. Examples of embedding compositions that can be used include polymeric substances and waxes. The active ingredient can also be in micro-encapsulated form, if appropriate, with one or more of the above-described excipients.

Liquid dosage forms useful for oral administration include pharmaceutically acceptable emulsions, lyophiles for reconstitution, microemulsions, solutions, suspensions, syrups and elixirs. In addition to the active ingredient, the liquid dosage forms may contain inert diluents commonly used in the art, such as, for example, water or other solvents, cyclodextrins and derivatives thereof, solubilizing agents and emulsifiers, such as ethyl alcohol, isopropyl alcohol, ethyl carbonate, ethyl acetate, benzyl alcohol, benzyl benzoate, propylene glycol, 1,3-butylene glycol, oils (in particular, cottonseed, groundnut, corn, germ, olive, castor and sesame oils), glycerol, tetrahydrofuryl alcohol, polyethylene glycols and fatty acid esters of sorbitan, and mixtures thereof.

Besides inert diluents, the oral compositions can also include adjuvants such as wetting agents, emulsifying and suspending agents, sweetening, flavoring, coloring, perfuming and preservative agents.

Suspensions, in addition to the active compounds, may contain suspending agents as, for example, ethoxylated isostearyl alcohols, polyoxyethylene sorbitol and sorbitan esters, microcrystalline cellulose, aluminum metahydroxide, bentonite, agar-agar and tragacanth, and mixtures thereof.

Formulations of the pharmaceutical compositions for administration to the mouth may be presented as a mouthwash, or an oral spray, or an oral ointment.

Formulations of the pharmaceutical compositions for rectal, vaginal, or urethral administration may be presented as a suppository, which may be prepared by mixing one or more active compounds with one or more suitable nonirritating excipients or carriers comprising, for example, cocoa butter, polyethylene glycol, a suppository wax or a salicylate, and which is solid at room temperature, but liquid at body temperature and, therefore, will melt in the rectum or vaginal cavity and release the active compound.

Formulations which are suitable for vaginal administration also include pessaries, tampons, creams, gels, pastes, foams or spray formulations containing such carriers as are known in the art to be appropriate.

Alternatively or additionally, compositions can be formulated for delivery via a catheter, stent, wire, or other intraluminal device. Delivery via such devices may be especially useful for delivery to the respiratory tract particularly the lungs.

Dosage forms for the topical administration include powders, sprays, ointments, pastes, creams, lotions, gels, solutions, patches and inhalants. The active compound may be mixed under sterile conditions with a pharmaceutically acceptable carrier, and with any preservatives, buffers, or propellants that may be required.

The ointments, pastes, creams and gels may contain, in addition to an active compound, excipients, such as animal and vegetable fats, oils, waxes, paraffins, starch, tragacanth, cellulose derivatives, polyethylene glycols, silicones, bentonites, silicic acid, talc and zinc oxide, or mixtures thereof.

Powders and sprays can contain, in addition to an active compound, excipients such as lactose, talc, silicic acid, aluminum hydroxide, calcium silicates and polyamide powder, or mixtures of these substances. Sprays can additionally contain customary propellants, such as chlorofluorohydrocarbons and volatile unsubstituted hydrocarbons, such as butane and propane.

Examples of suitable aqueous and nonaqueous carriers that may be employed in the pharmaceutical compositions of the invention include water, ethanol, polyols (such as glycerol, propylene glycol, polyethylene glycol, and the like), and suitable mixtures thereof, vegetable oils, such as olive oil, and injectable organic esters, such as ethyl olcate. Proper fluidity can be maintained, for example, by the use of coating materials, such as lecithin, by the maintenance of the required particle size in the case of dispersions, and by the use of surfactants.

These compositions may also contain adjuvants such as preservatives, wetting agents, emulsifying agents and dispersing agents. Prevention of the action of microorganisms may be ensured by the inclusion of various antibacterial and antifungal agents, for example, paraben, chlorobutanol, phenol sorbic acid, and the like. It may also be desirable to include isotonic agents, such as sugars, sodium chloride, and the like into the compositions. In addition, prolonged absorption of the injectable pharmaceutical form may be brought about by the inclusion of agents that delay absorption such as aluminum monostearate and gelatin.

For use in the methods of this invention, active compounds can be given per se or as a pharmaceutical composition containing, for example, 0.1 to 99.5% (more preferably, 0.5 to 90%) of active ingredient in combination with a pharmaceutically acceptable carrier.

Actual dosage levels of the active ingredients in the pharmaceutical compositions may be varied so as to obtain an amount of the active ingredient that is effective to achieve the desired therapeutic response for a particular patient, composition, and mode of administration, without being toxic to the patient.

The selected dosage level will depend upon a variety of factors including the activity of the particular compound or combination of compounds employed, or the ester, salt or amide thereof, the route of administration, the time of administration, the rate of excretion of the particular compound(s) being employed, the duration of the treatment, other drugs, compounds and/or materials used in combination with the particular compound(s) employed, the age, sex, weight, condition, general health and prior medical history of the patient being treated, and like factors well known in the medical arts.

A physician or veterinarian having ordinary skill in the art can readily determine and prescribe the therapeutically effective amount of the pharmaceutical composition required. For example, the physician or veterinarian could start doses of the pharmaceutical composition or compound at levels lower than that required in order to achieve the desired therapeutic effect and gradually increase the dosage until the desired effect is achieved. By “therapeutically effective amount” is meant the concentration of a compound that is sufficient to elicit the desired therapeutic effect. It is generally understood that the effective amount of the compound will vary according to the weight, sex, age, and medical history of the subject. Other factors which influence the effective amount may include, but are not limited to, the severity of the patient's condition, the disorder being treated, the stability of the compound, and, if desired, another type of therapeutic agent being administered with the compound of the invention. A larger total dose can be delivered by multiple administrations of the agent. Methods to determine efficacy and dosage are known to those skilled in the art (Isselbacher et al. (1996) Harrison's Principles of Internal Medicine 13 ed., 1814-1882, herein incorporated by reference).

In general, a suitable daily dose of an active compound used in the compositions and methods of the invention will be that amount of the compound that is the lowest dose effective to produce a therapeutic effect. Such an effective dose will generally depend upon the factors described above.

If desired, the effective daily dose of the active compound may be administered as one, two, three, four, five, six or more sub-doses administered separately at appropriate intervals throughout the day, optionally, in unit dosage forms. In certain embodiments of the present invention, the active compound may be administered two or three times daily. In preferred embodiments, the active compound will be administered once daily.

The patient receiving this treatment is any animal in need, including primates, in particular humans, and other mammals such as equines, cattle, swine and sheep; and poultry and pets in general.

In certain embodiments, compounds of the invention may be used alone or conjointly administered with another type of therapeutic agent. As used herein, the phrase “conjoint administration” refers to any form of administration of two or more different therapeutic compounds such that the second compound is administered while the previously administered therapeutic compound is still effective in the body (e.g., the two compounds are simultaneously effective in the patient, which may include synergistic effects of the two compounds). For example, the different therapeutic compounds can be administered either in the same formulation or in a separate formulation, either concomitantly or sequentially. In certain embodiments, the different therapeutic compounds can be administered within one hour, 12 hours, 24 hours, 36 hours, 48 hours, 72 hours, or a week of one another. Thus, an individual who receives such treatment can benefit from a combined effect of different therapeutic compounds.

In certain embodiments, conjoint administration of compounds of the invention with one or more additional therapeutic agent(s) (e.g., one or more additional chemotherapeutic agent(s)) provides improved efficacy relative to each individual administration of the compound of the invention or the one or more additional therapeutic agent(s). In certain such embodiments, the conjoint administration provides an additive effect, wherein an additive effect refers to the sum of each of the effects of individual administration of the compound of the invention and the one or more additional therapeutic agent(s).

This invention includes the use of pharmaceutically acceptable salts of compounds of the invention in the compositions and methods of the present invention. In certain embodiments, contemplated salts of the invention include, but are not limited to, alkyl, dialkyl, trialkyl or tetra-alkyl ammonium salts. In certain embodiments, contemplated salts of the invention include, but are not limited to, L-arginine, benethamine, benzathine, betaine, calcium hydroxide, choline, deanol, diethanolamine, diethylamine, 2-(diethylamino)ethanol, ethanolamine, ethylenediamine, N-methylglucamine, hydrabamine, 1H-imidazole, lithium. L-lysine, magnesium, 4-(2-hydroxyethyl)morpholine, piperazine, potassium, 1-(2-hydroxyethyl)pyrrolidine, sodium, triethanolamine, tromethamine, and zinc salts. In certain embodiments, contemplated salts of the invention include, but are not limited to, Na, Ca, K, Mg, Zn or other metal salts.

The pharmaceutically acceptable acid addition salts can also exist as various solvates, such as with water, methanol, ethanol, dimethylformamide, and the like. Mixtures of such solvates can also be prepared. The source of such solvate can be from the solvent of crystallization, inherent in the solvent of preparation or crystallization, or adventitious to such solvent.

Examples of pharmaceutically acceptable antioxidants include: (1) water-soluble antioxidants, such as ascorbic acid, cysteine hydrochloride, sodium bisulfate, sodium metabisulfite, sodium sulfite and the like: (2) oil-soluble antioxidants, such as ascorbyl palmitate, butylated hydroxyanisole (BHA), butylated hydroxytoluene (BHT), lecithin, propyl gallate, alpha-tocopherol, and the like; and (3) metal-chelating agents, such as citric acid, ethylenediamine tetraacetic acid (EDTA), sorbitol, tartaric acid, phosphoric acid, and the like.

In certain embodiments, the invention relates to a method for conducting a pharmaceutical business, by manufacturing a formulation of a compound of the invention, or a kit as described herein, and marketing to healthcare providers the benefits of using the formulation or kit for treating or preventing any of the diseases or conditions as described herein.

In certain embodiments, the invention relates to a method for conducting a pharmaceutical business, by providing a distribution network for selling a formulation of a compound of the invention, or kit as described herein, and providing instruction material to patients or physicians for using the formulation for treating or preventing any of the diseases or conditions as described herein.

In certain embodiments, the invention comprises a method for conducting a pharmaceutical business, by determining an appropriate formulation and dosage of a compound of the invention for treating or preventing any of the diseases or conditions as described herein, conducting therapeutic profiling of identified formulations for efficacy and toxicity in animals, and providing a distribution network for selling an identified preparation as having an acceptable therapeutic profile. In certain embodiments, the method further includes providing a sales group for marketing the preparation to healthcare providers.

In certain embodiments, the invention relates to a method for conducting a pharmaceutical business by determining an appropriate formulation and dosage of a compound of the invention for treating or preventing any of the disease or conditions as described herein, and licensing, to a third party, the rights for further development and sale of the formulation.

All publications and patents mentioned herein are hereby incorporated by reference in their entirety as if each individual publication or patent was specifically and individually indicated to be incorporated by reference. In case of conflict, the present application, including any definitions herein, will control. The compounds, synthetic methods, and experimental protocols and results of U.S. application Ser. No. 13/680,582, filed Nov. 19, 2012, are hereby incorporated by reference.

While specific embodiments of the subject invention have been discussed, the above specification is illustrative and not restrictive. Many variations of the invention will become apparent to those skilled in the art upon review of this specification and the claims below. The full scope of the invention should be determined by reference to the claims, along with their full scope of equivalents, and the specification, along with such variations.

What is claimed is:

1. A compound, comprising: a scaffold having a structure of the formula:

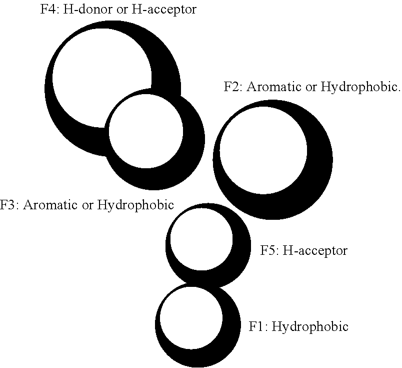

2. The compound of claim 1, wherein the scaffold is hydrophilic.

3. The compound of claim 1, wherein a plurality of pharmacophores are hydrophobic.

4. The compound of claim 1, wherein each branch terminates in a pharmacophore.

5. The compound of claim 1, wherein every pharmacophore is identical.

6. The compound of claim 1, wherein each branch is hydrophilic.

7. A pharmaceutical composition, comprising a compound of claim 1 and one or more pharmaceutically acceptable excipients.
